(19) United States
(12) Patent Application Publication
(10) Pub. No.: US 2017/0136060 A1
(43) Pub. Date: May 18, 2017

(54) TREATMENT OF VAGINITIS

(71) Applicant: Glock Health, Science and Research GmbH, Deutsch Wagram (AT)

(72) Inventor: Gaston GLOCK, Velden (AT)

(21) Appl. No.: 15/349,727

(22) Filed: Nov. 11, 2016

(30) Foreign Application Priority Data
Nov. 12, 2015 (EP) 15194288.5

(51) Int. Cl. A61K 33/06; A61K 45/06; A61K 9/14

(52) U.S. Cl. CPC A61K 33/06; A61K 9/14; A61K 45/06

(57) ABSTRACT

Treatment of vaginal mycoses, bacterial vaginoses, and other forms of the vaginitis (inflammation of the vagina) by clinoptilolite having a particle size of between 0.2 and 10 μm. Clinoptilolite, when used externally, is effective in the treatment of these vaginal disorders in mammals and humans, and also for restoring a healthy vaginal microbiota. The clinoptilolite may be used with one or more of the following adjuvants: pharmaceutically acceptable carrier materials, viable microorganisms and/or extracts thereof, nutrients for the healthy vaginal microbiota (e.g. lactose, etc.), and/or substances which favorably influence the vaginal environment for the healthy vaginal microbiota (e.g. estradiol, organic acids, etc.). The composition used may be applied locally, preferably in one of the following administration forms: foam, suppository, vaginal tablet, ovule, gel, aerosol, powder, rinse, douche, cream/ointment, or suspension.

The invention relates to the treatment of vaginal mycoses, bacterial vaginoses, and other forms of vaginitis (inflammation of the vagina) in accordance with claim 1.

Known from WO 00/64586 is an apparatus and an associated method for comminuting mineral material, for which, subsequently, there is a cursory listing of the diseases in which it can be successfully used, with virtually no disease being left out. Several references were made to tests relating to carcinogenic disorders, liver disorders, diabetes, and multiple sclerosis, without any indication of the testing organization or any particulars at all. Other fields of application mentioned are the production of foods, of cosmetics, of crop protection products, of cigarettes, and raw materials in the construction industry. The apparatus described is capable of comminuting more than 98% of all the particles below 4.3 μm, with more than 28% of all the particles being smaller than 0.5 μm. There is no relation between these details and those of the application.

Hailing from the same inventor is WO 2007/054085, which discloses clinoptilolite comminuted to below 100 nanometers, corresponding to 0.1 μm, with propolis and/or colostrum as an antiviral agent, for the treatment of conditions including influenza, cold, coughing, measles, mumps, rubella, slapped cheek syndrome, three-day fever, chickenpox, Pfeiffer's glandular fever, SARS, zytomegalo virus, diarrhea, hepatitis, polio, herpes labialis, warts, rabies, Lassa fever, Ebola, Marburg fever, hantavirus fever, FSME, RSSE, Louping-ill encephalitis, Powassan encephalitis, Kyasanur forest fever, Omsk hemorrhagic fever, Colorado tick fever, yellow fever, Dengue fever, Japanese encephalitis, West Nile fever, Chikungunya fever, Q′nyong-nyong fever, Rift valley fever, sandfly fever, Ross river fever, Sindbis fever, Mayaro fever, Murray valley encephalitis, St. Louis encephalitis, Rocio encephalitis, California encephalitis, Bunyamwera fever, Oropouche fever, AIDS, herpes genitalis and/or herpes simplex. As part of the “treatment” of AIDS, one of the clinical pictures occurring, among many others, is said to be vaginal candidiasis. No clinical tests are mentioned, and everything is pure wishful thinking.

WO 2008/003101, from the inventor of the present application, discloses a method for purifying clinoptilolite to remove heavy metals.

WO 2010/057849 describes the improvement in wound healing from application of comminuted clinoptilolite, which reduces the concentration of amines. Without more detailed reference to the individual applications, particle sizes of 2 to 16 μm are stated, which are also required to possess a specified zeta potential and a specified specific surface area; no measurement methods are stated. Examples indicated are exclusively in vitro experiments with artificially created, amine-containing aqueous solutions.

From EP 956 858 it is known that probiotic bacteria are useful in the treatment of vaginal infections.

In general the following may be observed:

In the typical (healthy) case, the vaginal microbiota of the adult woman is made up very largely of various lactobacilli (depending on ethnicity, Lactobacillus crispatus, L. gasseri, L. iners, L. jensenii), which among other things ensure an optimum pH and suppress the growth of pathogens. Under the influence of various environmental factors, the natural vaginal flora is attacked and, as a consequence of an imbalance (dysbiosis), there may be a prevalence of pathogenic microbes.

The aim and object of the invention is to specify a treatment which firstly controls the pathogenic microbes as effectively as possible and secondly as far as possible preserves the useful lactobacilli.

This object according to the invention is achieved through the features specified in claim 1; in other words, clinoptilolite having a particle size of between 0.2 and 10 μm is used locally (externally) for the treatment of vaginal disorders such as bacterial vaginosis, vulvovaginal candidiasis, and trichomoniasis of mammals and humans, and also for restoring the healthy vaginal microbiota. For jurisdictions in which this is possible or acquired, the invention consists in using clinoptilolite having a particle size of between 0.2 and 10 μm for producing a medicinal product for the treatment of the disorder(s) identified at the outset.

The clinoptilolite has preferably been freed from heavy metals, more preferably by a method in accordance with the methods indicated in EP 2 040 837 or the corresponding U.S. Pat. No. 8,173,101. For jurisdictions in which this is possible, the disclosure content of these publications is made part of the content of the present patent application by reference.

The effectiveness of the substance according to the invention is evident from the investigations and experiments, outlined below, on a number of different microorganism strains. It is demonstrated that the substance has a growth-inhibiting effect on members of the genus Candida and also on selected bacterial pathogens of the vaginal flora, while representatives of a healthy vaginal flora are in fact promoted in their growth.

In order to illustrate the connection between the particle size and the activity, corresponding comparative experiments are appended at the end of the experiments demonstrating the activity.

The indications placed in [n] refer to the references given at the end of the description. The results are represented in various tables and graphs in the form of figures, where:

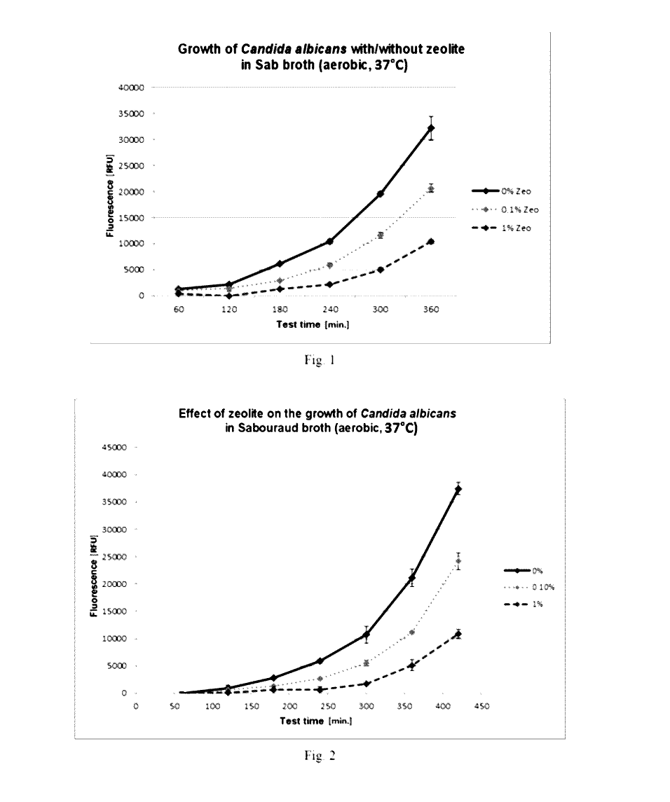

FIG. 1 shows the growth of Candida albicans with/without zeolite in Sab broth,

FIG. 2 shows the effect of zeolite on the growth of Candida albicans in Sabouraud broth,

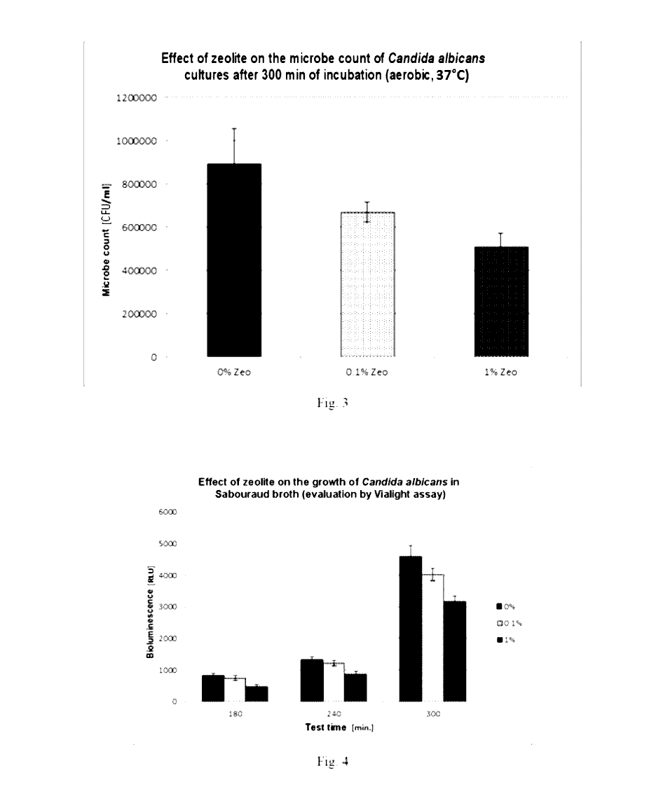

FIG. 3 shows the effect of zeolite on the microbe counter Candida albicans cultures after 300 min of incubation,

FIG. 4 shows the effect of zeolite on the growth of Candida albicans in Sabouraud broth (Vialight assay),

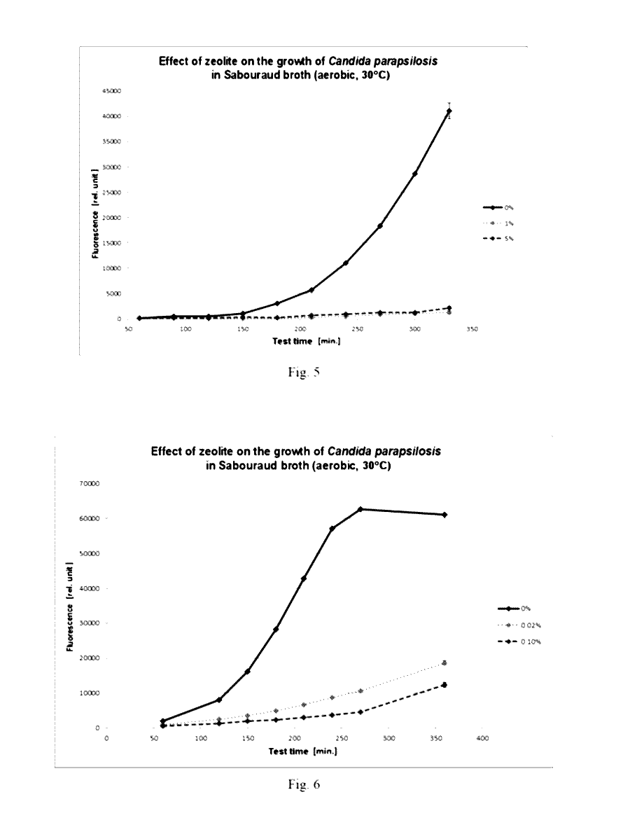

FIG. 5 shows the effect of zeolite on the growth of Candida parapsilosis in Sabouraud broth,

FIG. 6 shows the effect of zeolite on the growth of Candida parapsilosis in Sabouraud broth,

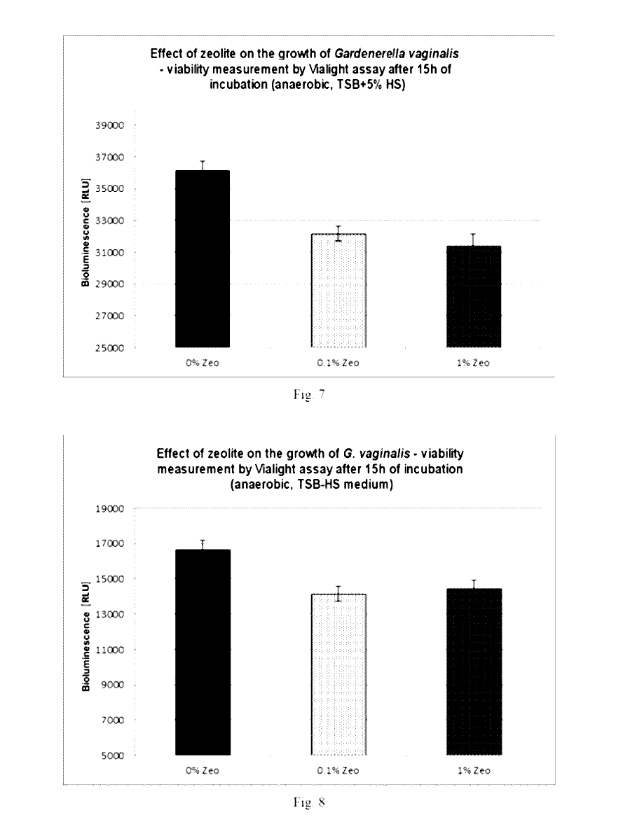

FIG. 7 shows the effect of zeolite on the growth of Gardnerella vaginalis viability measurement by Vialight assay after 15 hours of incubation,

FIG. 8 shows the effect of zeolite on the growth of G. vaginalis viability measurement by Vialight assay after 15 hours of incubation,

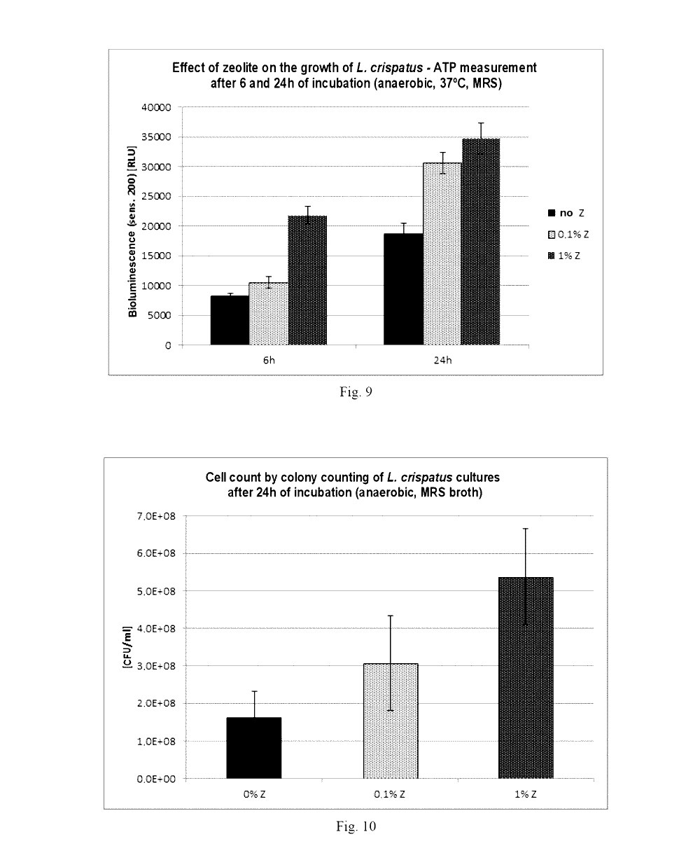

FIG. 9 shows the effect of zeolite on the growth of L. crispatus—ATP measurement after 6 and 24 hours of incubation,

FIG. 10 shows the cell count of L. crispatus cultures after 24 hours of incubation, determined by colony counting,

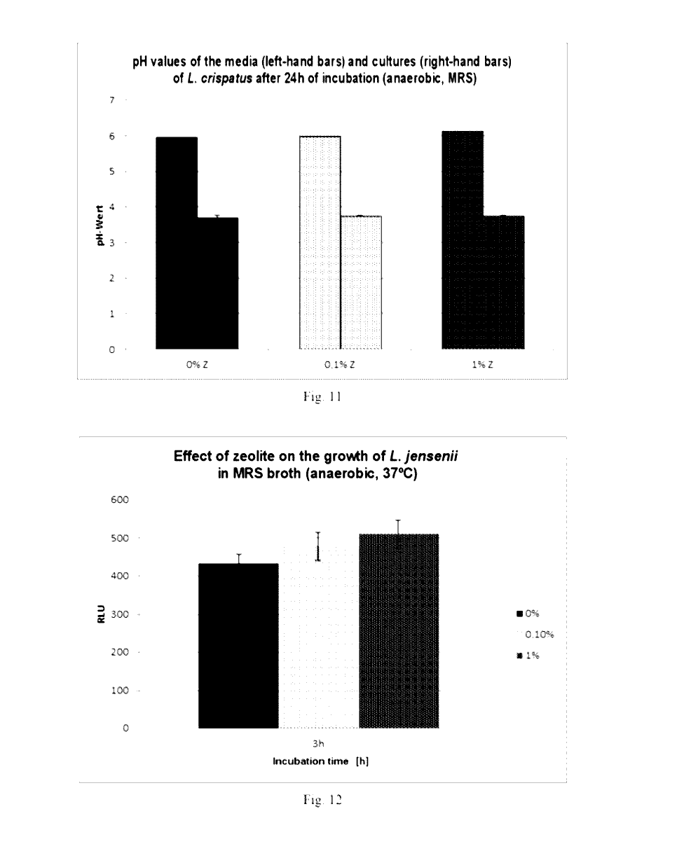

FIG. 11 shows the pH values of the media and cultures of L. crispatus after 24 hours of incubation,

FIG. 12 shows the effect of zeolite on the growth of L. jensenii in MRS broth within 3 hours,

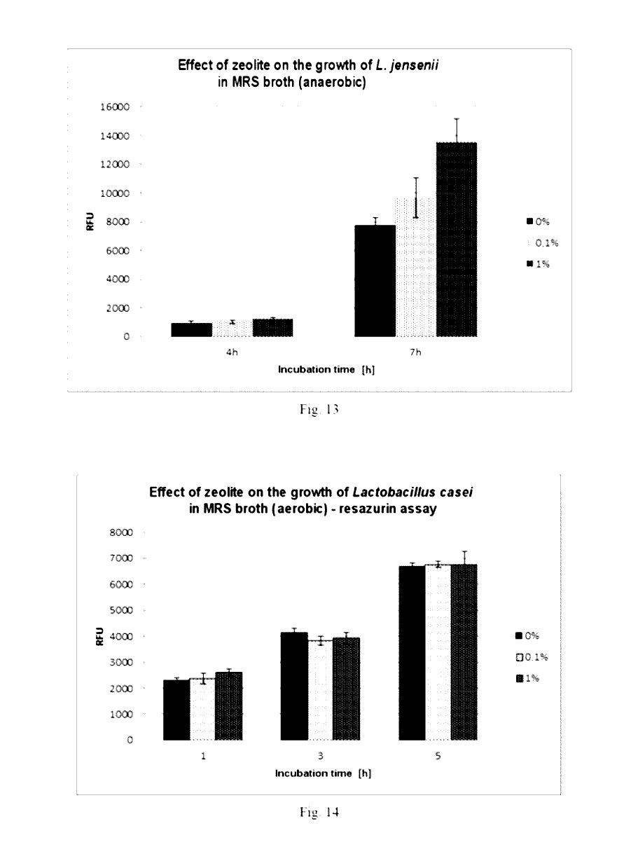

FIG. 13 shows the effect of zeolite on the growth of L. jensenii in MRS broth within 4 and 7 hours,

FIG. 14 shows the effect of zeolite on the growth of Lactobacillus casei in MRS broth—resazurin assay,

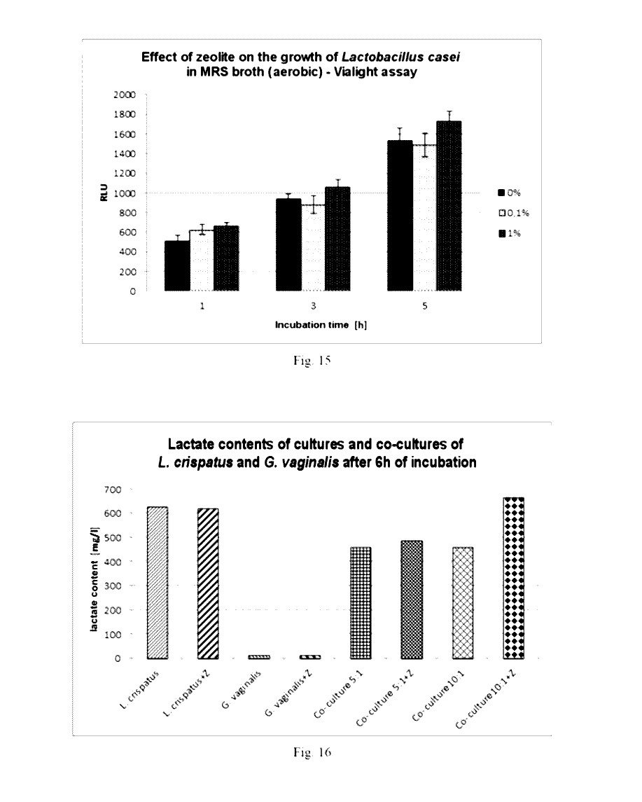

FIG. 15 shows the effect of zeolite on the growth of Lactobacillus casei in MRS broth—Vialight assay,

FIG. 16 shows the lactate contents of various cultures and co-cultures after 6 hours of incubation,

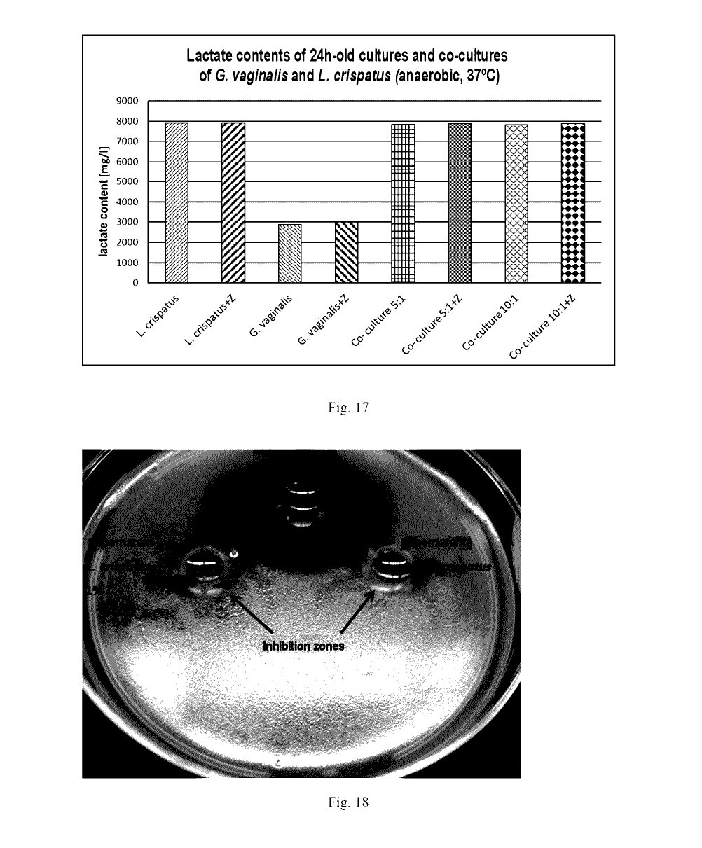

FIG. 17 shows the lactate contents of various 24-hour-old cultures and co-cultures,

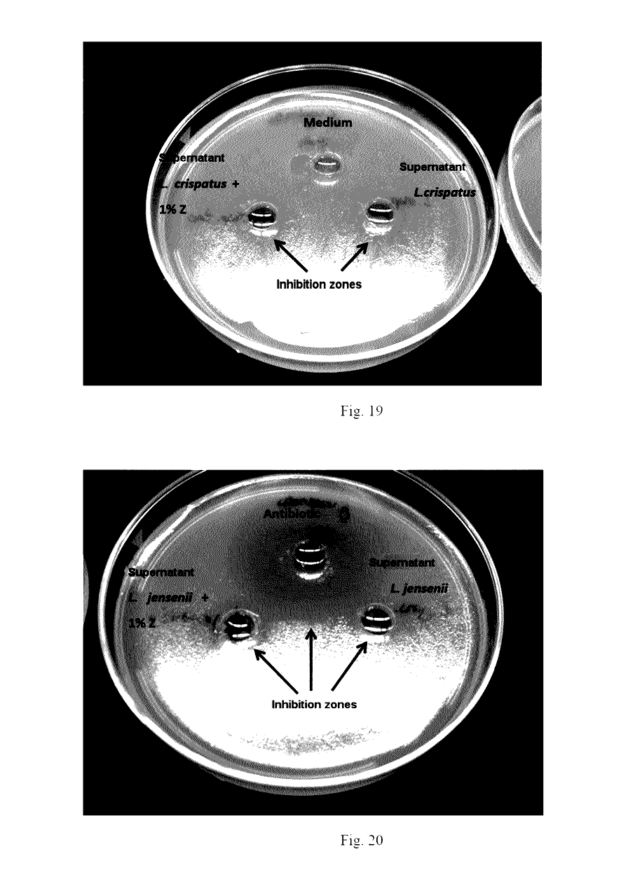

FIGS. 18-20 show agar plates with different cultures,

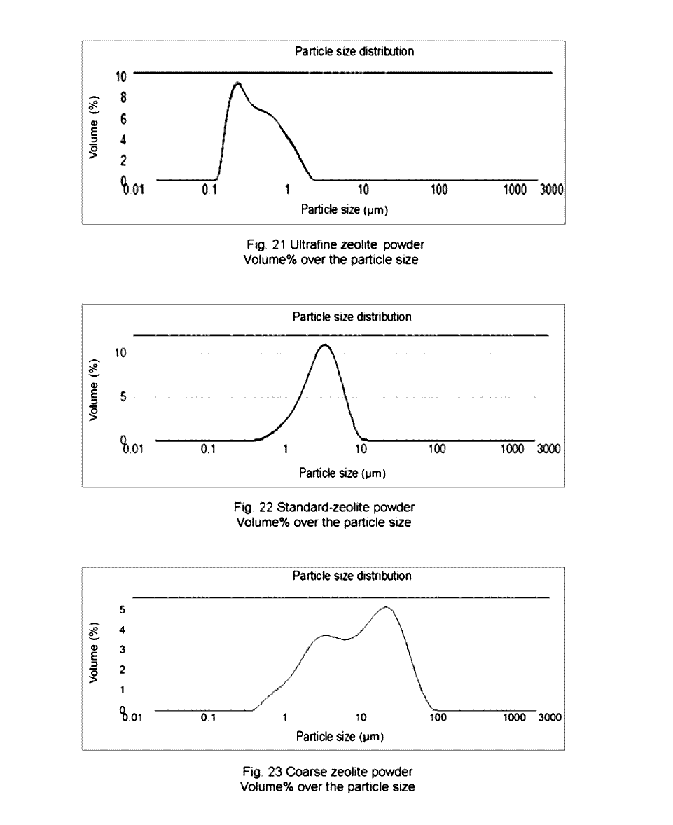

FIGS. 21-23 show particle size distributions used in the comparative experiments,

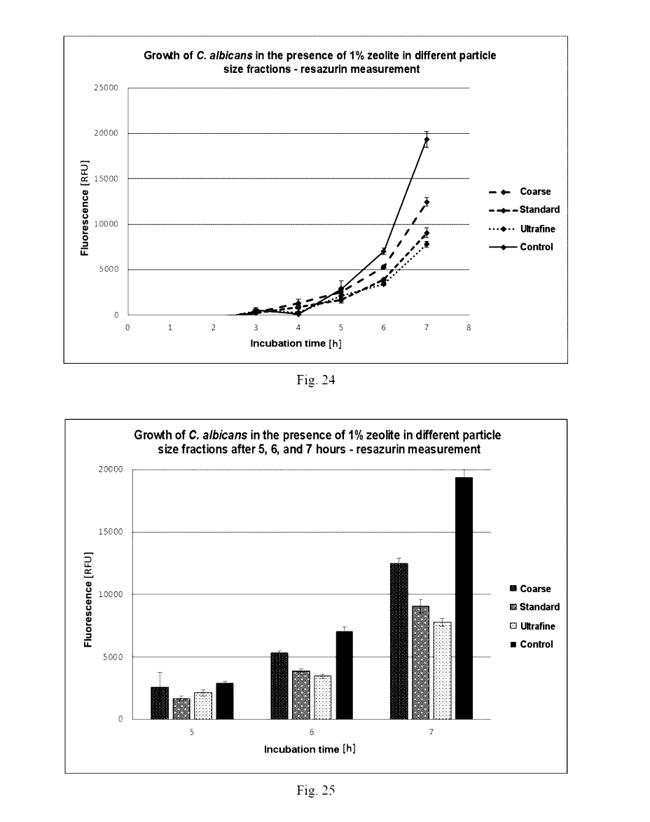

FIGS. 24 and 25 show the particle size distribution-dependent growth of C. albicans—resazurin measurement,

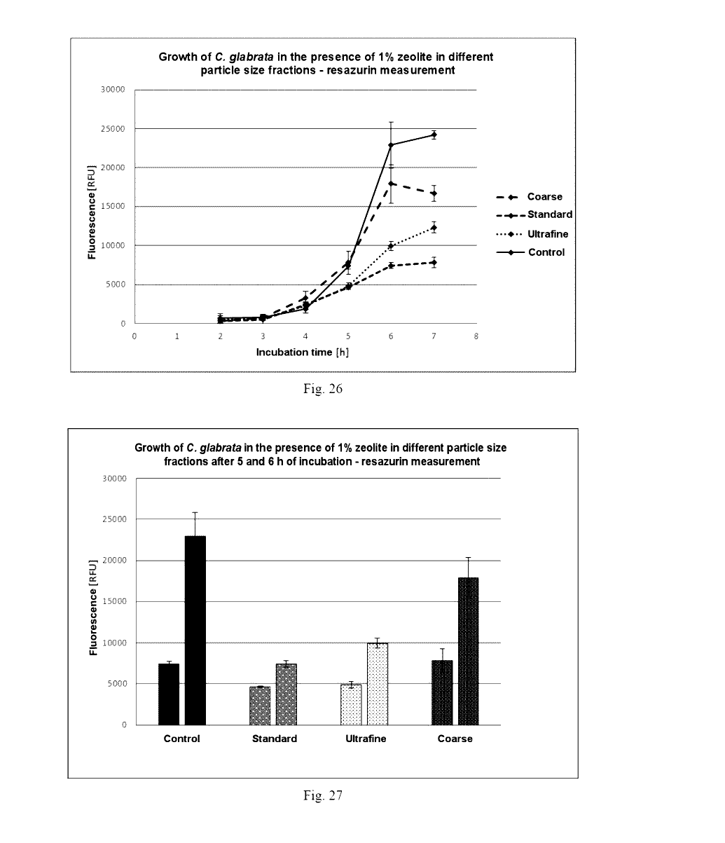

FIGS. 26-28 show the same for C. glabrata and

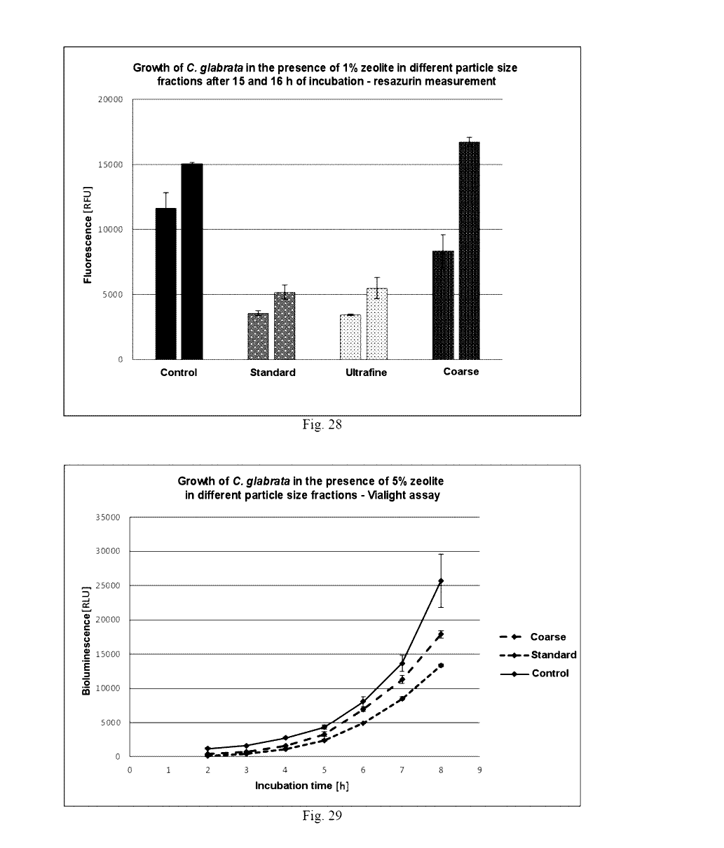

FIG. 29 shows the particle size distribution-dependent growth of C. glabrata—Vialight assay.

### THE DISORDERS AND THEIR TREATMENT

Vaginal Mycosis:

Vaginal mycosis represents the second most common cause, after bacterial vaginosis, of an infection of the vagina.

The main causative organisms of confirmed infections are yeasts of genus Candida, with Candida albicans being responsible for 85-90% of all vaginal fungal infections.

Risk factors which promote vaginal mycosis are:

Pregnancy

Consumption of antibiotics

Diabetes mellitus

Immunosuppressants

Cancer therapy drugs

Drugs containing cortisone

Stress or mental loading

Recommended therapies in the prior art lie in the topical application of various antimycotics such as polyenes (nystatin), imidazoles (butoconazoles, clotrimazole, etc.) or ciclopirox olamine. They are administered intravaginally in a large number of different preparations such as vaginal tablets, ovules or vaginal creams. The duration of treatment may be 1 to 7 days. A further possibility lies in the single administration of an active triazole ingredient (fluconazole, itraconazole). In parallel with the antimycotic therapy, the local administration of probiotics (e.g., Gynoflor, Multi-Gyn Flora plus, and many more) may support the re-establishment of the healthy vaginal flora and the lowering of the local pH. These products (vaginal tablets, ovules, gels, creams, etc.) may inter alia contain either viable lactobacilli (“Doderlein bacilli”) or lysates thereof, and also estriol, which as an estrogen derivative promotes the development of a “healthy” mucosal environment, and lactose as a nutrient for the lactobacilli.

In the case of the local immunological deficiency, there is a relapse just a short time after the end of therapy. Against this chronically recurring Candida albicans vulvovaginitis, in the absence of alternatives, local or oral maintenance therapies are recommended in order to avoid relapses. Here, as an initial therapy, a topical product as described above is applied for 7-14 days, and in parallel with this, orally, in the first week, 3×200 mg of fluconazole are administered. The maintenance therapy consists subsequently of a weekly administration of 150 mg of fluconazole.

All concentration figures in the description, the claims, and the figures should be viewed, unless otherwise stated, as mg/ml or as % M/V [kg/liter].

In accordance with the invention, clinoptilolite having a particle size of between 0.2 and 10 μm is applied locally in the concentration of 1 mg/ml to 10 mg/ml {corresponding to 0.1% M/V to 1% M/V, when M is given in kg and V in liters}, with the following effect on Candida albicans:

### 1^{st} Experiment:

Candida albicans (DSM No. 1386) [4] was cultivated, in accordance with methods frequently used routinely, on yeast glucose chloramphenicol (YGC) agar and thereafter transferred to liquid medium (Sabouraud broth).

Following determination of the cell count using a Thoma counting chamber, 12 experimental cultures were produced, each with 1×10⁵ cells/ml, with no zeolite powder being added to four cultures (control), zeolite powder in an amount of 1 mg/ml being added to four cultures and zeolite powder in an amount of 10 mg/ml being added to a further four cultures. The experimental cultures were incubated at 37° C. under aerobic conditions with shaking.

For determination of the blank values, the experiment was also run with all the media without microbes.

Over the period of six hours, aliquots of the cultures were taken hourly for determining the metabolic activity by means of viability assay (resazurin assay) [7].

The results, from which the high activity is immediately apparent, are illustrated in graph form in FIG. 1; curves from top to bottom: 0%; 0.1% and 1% zeolite.

### 2^{nd} Experiment:

The experiment described above was repeated, with the following two changes:

The experiment time was extended to seven hours

Aliquots of the cultures were diluted after five hours of incubation for determination of the actual cell count, then plated out onto Sabouraud agar and evaluated by colony counting after incubation.

FIG. 2 shows the results of the resazurin assay; curves as in FIG. 1. The result of the preceding growth experiment was confirmed. Represented in graph form in FIG. 3 are the empirically determined microbe counts of the experimental groups (from left: 0%, 0.1% and 1% zeolite).

### 3^{rd} Experiment:

In a further repetition of the experiment, an alternative measurement of the metabolic activity (Vialight assay) [8] was selected in order to reinforce the results of the experiments. FIG. 4 (columns, in each case from left: 0, 0.1% and 1% zeolite) shows the experimental groups after 3, 4 and 5 hours of incubation. As can be seen, it was again possible with the substance according to the invention to achieve a reduction of the metabolic activity of Candida albicans in broth culture.

The effect of the substance according to the invention was subsequently tested in concentrations of 10 mg/ml and 50 mg/ml on Candida parapsilosis (in-house isolate; S 0811-01) [9]:

Candida parapsilosis was first cultured, in accordance with methods frequently used in routine practice, on YGC agar, and subsequently transferred to a liquid medium (Sabouraud broth) and incubated.

Following determination of the cell count using a counting chamber, 9 experimental cultures (yeast cultures) were produced each with 1×10⁶ yeast cells/ml, with no zeolite powder being added to 3 cultures (control), zeolite powder in an amount of 10 mg/ml (1% M/V) being added to 3 cultures, and zeolite powder in an amount of 50 mg/ml (5% M/V) being added to a further 3 cultures. The experimental cultures were incubated at 30° C. with shaking:

3×12 ml Sabouraud broth

3×12 ml Sabouraud broth, 1% zeolite

3×12 ml Sabouraud broth and 5% zeolite

For determination of the blank values, aliquots of all the media were incubated without microbes as well, and the experiment was run with these aliquots also.

After 60 min, 120 min, and then every further 30 min, the metabolic activities of each experimental culture were determined indirectly using a common viability test (resazurin assay). The measurements for the three cultures of the same conditions were averaged and have been shown, including the standard deviation, in FIG. 5 (curves, from the top: 0 mg/ml, 10 mg/ml and 50 mg/ml).

As can be seen, both of the doses used of the substance according to the invention resulted in a significant inhibition of growth of the experimental microbe.

### 4^{th} Experiment:

The effect of the substance according to the invention in concentrations of 1 mg/ml and 0.2 mg/ml on Candida parapsilosis:

Candida parapsilosis was first cultured, in accordance with methods frequently used in routine practice, on YGC agar, and subsequently transferred to a liquid medium (Sabouraud broth) and incubated.

Following determination of the cell count using a Neubauer counting chamber, 9 experimental cultures (yeast cultures) were produced each with 1×10⁶ yeast cells/ml, with no zeolite powder being added to 3 cultures (control), zeolite powder in an amount of 1 mg/ml (0.1% M/V) being added to 3 cultures, and zeolite powder in an amount of 0.2 mg/ml (0.02% M/V) being added to a further 3 cultures. The experimental cultures were incubated at 30° C. with shaking:

3×12 ml Sabouraud broth

3×12 ml Sabouraud broth, and 0.1% zeolite

3×12 ml Sabouraud broth and 0.02% zeolite

For determination of the blank values, aliquots of all the media were incubated without microbes as well, and the experiment was run with these aliquots also.

After 60 min, 120 min, and then every further 30 min, the metabolic activities of each experimental culture were determined indirectly using a common viability test (resazurin assay). The measurements for the three cultures of the same conditions were averaged and have been shown, including the standard deviation, in FIG. 6 (curves, from the top: 0%, 0.02% and 0.1% zeolite).

Even at the lowest concentration used (0.02%), middle line in FIG. 6, the substance according to the invention has a significantly growth-retarding effect on Candida parapsilosis.

### Bacterial Vaginosis

Bacterial vaginosis represents the most common form of vaginitis.

It occurs when the healthy H₂O₂-producing vaginal flora (primarily various representatives of the lactobacilli) are replaced, in a dysbiosis, by predominantly anaerobic bacteria. These bacteria include predominantly Gardnerella vaginalis and Atopobium vaginae, but also representatives of the genera Megasphaera, Dialister, Mobiluncus, Prevotella, and others. Risk factors which promote bacterial vaginosis include the following:

Low socioeconomic status (->stress)

Frequent vaginal douching

Smoking

Unprotected sexual relations with frequently changing partners

Contraceptives in the uterus

Frequent use of relatively high doses of the spermicide Nonoxynol-9

### Recommended Therapies:

Outside of pregnancy, a 7-day therapy is carried out using Metronidazol (2×500 mg/day). Other possibilities lie in the local administration of Metronidazol or Clindamycin in the form of 2% vaginal creams (duration: 7 days). Also possible is the use of vaginal tablets for twice-daily local administration of 500 mg of Metronidazol for a duration of 7 days. There are also ovules for the intravaginal administration of Clindamycin (100 mg daily for 3 days).

The therapies described do not, however, eliminate the adhering bacterial biofilm, and hence the cure rate after 3 months is only 60-70%, and even lower after 6 months. Through the use of probiotics, whose effects include lowering of the vaginal pH and promotion of the healthy vaginal flora, it is possible to reduce the rate of recurrence of BV by around half (see [2]: S1 therapy of the vaginal mycosis).

The effectiveness of the substance according to the invention is evident from the experiments and investigations described below on Gardnerella vaginalis:

### 5^{th} Experiment:

Effect of the substance according to the invention at concentration of 1 mg/ml (0.1% M/V) and 10 mg/ml (1% M/V) on Gardnerella vaginalis: Gardnerella vaginalis (DSM No. 4944) [5] was cultured, in accordance with methods frequently used in routine practice, on Casman agar and subsequently transferred to liquid medium (TSB+5% horse serum).

Following determination of the cell count using a Thoma counting chamber, 12 experimental cultures were produced each with 1×10⁵ cells/ml, with no zeolite powder added to 4 cultures (control), zeolite powder in an amount of 1 mg/ml being added to 4 cultures, and zeolite powder in an amount of 10 mg/ml being added to a further 4 cultures. The experimental cultures were incubated at 37° C. under anaerobic conditions for 15 hours, with shaking.

For the determination of the blank values, the experiment was also run on all the media without microbes as well.

Subsequently, aliquots of the cultures were taken for determination of the metabolic activity by means of viability testing (Vialight assay).

The results are shown in graph form in FIG. 7 (bars, from left: 0%, 0.1% and 1% zeolite).

### 6^{th} Experiment:

The growth test was repeated with an even lower starting cell count (5×10⁴ cells/ml), and gave a comparable result; see FIG. 8 (bars, from left: 0%, 0.1% and 1% zeolite).

The promotion by the substance according to the invention of the growth of the healthy vaginal flora is evident from investigations and experiments depicted below which were carried out on bacteria of the genus Lactobacillus:

### 7^{th} Experiment:

Lactobacillus crispatus (DSM No. 20584) [6] was cultured anaerobically, in accordance with methods frequently used in routine practice, on de Man Rogosa Sharp (MRS) agar, and subsequently transferred to a liquid medium (MRS broth).

Following determination of the cell count using a Thoma counting chamber, 12 experimental cultures were produced each with 1×10⁶ cells/ml, with no zeolite powder being added to 4 cultures (control), zeolite powder in an amount of 1 mg/ml (0.1% M/V) being added to 4 cultures, and zeolite powder in an amount of 10 mg/ml (1% M/V) being added to a further 4 cultures. The experimental cultures were incubated at 37° C. under anaerobic conditions with shaking.

For the determination of the blank values, the experiment was also run with all of the media without microbes as well.

After 6 and 24 hours, aliquots of the cultures were taken for determination of the metabolic activity by viability testing (Vialight assay); see FIG. 9 (bars, from left, in each case: 0%, 0.1% and 1% zeolite). Additionally, after 24 hours, aliquots of the cultures were diluted and plated out onto MRS agar; see FIG. 10 (bars, from left: 0%, 0.1% and 1% zeolite); and the pH values of the experimental cultures were measured at the end of the experiment by a pH electrode; see FIG. 11 (bars, in each case: media on left, cultures on right, in each case from the left: 0%, 0.1% and 1% zeolite).

The results of colony counting (FIG. 10) confirmed the viability measurements; FIG. 11 shows the pH values of the cultures after 24 h of incubation. Irrespective of whether the substance according to the invention was or was not added, the microbe reduced the pH of the medium to below 3.8 in all cases.

### 8^{th} Experiment:

Lactobacillus jensenii (DSM No. 20557) [12] was cultured anaerobically, in accordance with methods frequently used in routine practice, on de Man Rogosa Sharp (MRS) agar, and subsequently transferred to a liquid medium (MRS broth).

Following determination of the cell count using a Thoma counting chamber, 12 experimental cultures were produced each with 5×10⁵ cells/ml, with no zeolite powder being added to 4 cultures (control), zeolite powder in an amount of 1 mg/ml (0.1% M/V) being added to 4 cultures, and zeolite powder in an amount of 10 mg/ml (1% M/V) being added to a further 4 cultures. The experimental cultures were incubated at 37° C. under anaerobic conditions with shaking.

For the determination of the blank values, the experiment was also run with all of the media without microbes as well.

After 3 hours, aliquots of the cultures were taken for determination with the metabolic activity by viability testing (Vialight assay). FIG. 12 shows in graph form the result of the experiment. An increase in the metabolic performance is evident through incubation with the substance according to the invention.

### 9^{th} Experiment:

The growth experiment was repeated with the incubation time extended. The metabolic activity was measured (Vialight assay) after 4 and 7 hours in the same way as described above. FIG. 13 shows the analysis of the measurement results in graph form. The growth-promoting effect of the substance according to the invention on L. jensenii is even more clearly visible after 7 hours than after 3 hours in experiment 8.

### 10^{th} Experiment:

Lactobacillus casei (DSM No. 20011) [11] was cultured aerobically, in accordance with methods frequently used in routine practice, on de Man Rogosa Sharp (MRS) agar, and subsequently transferred to a liquid medium (MRS broth).

Following determination of the cell count using a Thoma counting chamber, 12 experimental cultures were produced each with 5×10⁵ cells/ml, with no zeolite powder being added to 4 cultures (control), zeolite powder in an amount of 1 mg/ml (0.1% M/V) being added to 4 cultures, and zeolite powder in an amount of 10 mg/ml (1% M/V) being added to a further 4 cultures. The experimental cultures were incubated at 37° C. under aerobic conditions with shaking.

For the determination of the blank values, the experiment was also run with all of the media without microbes as well.

After 1, 3, and 5 hours, aliquots of the cultures were taken for determination of the metabolic activity by viability testing (resazurin assay and Vialight assay); (see FIG. 14 and FIG. 15; both bars, from left: 0%, 0.1% and 1% zeolite).

The results in FIGS. 14 and 15 show, surprisingly, no effect at all of the substance according to the invention on the metabolic activity of the experimental microbe within the test time. Over wide sections of the experiment, it was possible neither by resazurin assay nor by ATP assay to measure significant differences between the individual conditions.

It is surprising that the substance according to the invention evidently and quite specifically promotes the species L. crispatus and L. jensenii, which are relevant to a healthy vaginal flora, in their growth, but at the same time exerts no influence on the growth of the species L. casei, which are not relevant for the vaginal flora.

The following investigations and experiments demonstrate that the substance according to the invention, in combination with a vaginal Lactobacillus strain, promotes the regeneration of the healthy vaginal environment and acts growth-inhibitingly on Gardnerella vaginalis, one of the principal pathogens of vaginosis.

### 11^{th} Experiment:

Lactobacillus crispatus (DSM No. 20584) [6] and Gardnerella vaginalis (DSM No. 4944) [5] were first cultivated in pure culture in accordance with methods frequently used in routine practice.

Following determination of the cell count of the pre-cultures using a Thoma counting chamber, 12 experimental cultures (cultures and co-cultures) each with 5×10⁵ cells/ml were produced in a mixed medium (TSB+HS and MRS in a ratio of 7+3):

1×10 ml of medium (7 ml TSB+5% horse serum and 3 ml of MRS broth)

1×G. vaginalis

1×G. vaginalis+0.5% zeolite

1×L. crispatus

1×L. crispatus+0.5% zeolite

1×L. crispatus+G. vaginalis (5:1) A

1×L. crispatus+G. vaginalis+0.5% zeolite (5:1) A

1×L. crispatus+G. vaginalis (5:1) B

1×L. crispatus+G. vaginalis+0.5% zeolite (5:1) B

1×L. crispatus+G. vaginalis (10:1) A

1×L. crispatus+G. vaginalis+0.5% zeolite (10:1) A

1×L. crispatus+G. vaginalis (10:1) B

1×L. crispatus+G. vaginalis+0.5% zeolite (10:1) B

The experimental cultures were incubated on an orbital shaker under anaerobic conditions at 37° C. After 6 and 24 h, aliquots of the cultures were centrifuged off (5 min, 15000 g), the supernatants were deactivated in a water bath at 80° C. for 15 min, and were passed on for enzymatic quantification of lactic acid [13]. The lactic acid produced by the vaginal Lactobacilli represents one of the main factors of the healthy vaginal environment.

The measurement results after 6 h of incubation are shown in graph form in FIG. 16. The values of the co-cultures were averaged in each case for the graph.

From FIG. 16 it is clear that the addition of the substance according to the invention to the co-cultures promotes the production of lactic acid by L. crispatus.

In the saturated cultures after an incubation time of 24 h, all of the co-cultures have approximately equal lactate contents to those of the L. crispatus pure cultures (see FIG. 17), an indication that this microbe was a determinant of the environment.

### 12^{th} Experiment:

Lactobacillus crispatus (DSM No. 20584) [6] and L. jensenii (DSM No. 20557) [12] were cultured anaerobically in liquid medium in accordance with methods frequently used in routine practice. The nutrient medium employed was MRS broth, with and without addition of the substance according to the invention (10 mg/ml), which had been preconditioned in the same medium before the experiment. Aliquots of the saturated cultures were centrifuged off and the supernatants were tested as described below for their growth-inhibiting effect on Gardnerella vaginalis.

G. vaginalis was grown overnight in TSB+5% horse serum and plated out densely onto BHI+5% horse serum agar plates. Then three holes were punched into each of the plates using a sterile pipette tip. The supernatants of the Lactobacillus cultures were pipetted into these recesses in the agar plates. The positive control used was a mix of antibiotics (penicillin/streptomycin), while medium (centrifuged MRS broth) was employed as negative control. The plates were incubated anaerobically at 37° C. until lawn growth of G. vaginalis was detectable. In order to raise the atmospheric humidity, wet cloths were added to the incubation containers.

On evaluation, an inhibition of the growth of Gardnerella vaginalis was evident in the form of zones of inhibition visible around the recesses containing the culture supernatants. This effect can be found in the case of both Lactobacillus strains tested, with the supernatants both with and without addition of the substance according to the invention, but is more strongly pronounced with L. crispatus than with L. jensenii. The figures below (FIGS. 18, 19 and 20) show by way of example the agar plates at the time of evaluation of the experiments. The dense bacterial lawn of Gardnerella vaginalis is recessed around the supernatants of the Lactobacillus cultures (and of the antibiotics run as a positive control).

In summary it can be stated that, firstly, it has been possible to show inhibition or negative influencing of the growth of potentially pathogenic vaginal microbes by the substance according to the invention. Secondly it has been possible to record that relevant microbes of the healthy vaginal microbiota are not only not impaired in their growth but are in fact promoted in their growth. This growth-inhibiting effect of the substance according to the invention on pathogenic vaginal microbes, accompanied by promotion tendency and specific promotion of the healthy vaginal microbiota, may be regarded as surprising and novel.

With regard to the effect of the particle size distribution of the zeolite powder according to the invention, three different batches were produced for the comparative experiments which are described below. Here, the distribution in line with FIG. 21 is referred to as “ultrafine zeolite powder”, the distribution in line with FIG. 22 as “standard zeolite powder”, and the distribution in line with FIG. 23 as “coarse zeolite powder”. The tabular data in this regard are as follows:

### “Ultrafine Zeolite Powder”

| | d (0.1) | d (0.5) | d (0.9) | |
|---|---|---|---|---|
| | [μm] | [μm] | [μm] | Residual |
| MW | 0.19 | 0.38 | 1.06 | 5.36 |
| S | 0.00 | 0.00 | 0.01 | 0.14 |
| RSD | 0.54 | 1.13 | 1.37 | 2.69 |

### “Standard Zeolite Powder” (as Also Used in Experiments 1-12)

| 008-02- | d (0.1) | d (0.5) | d (0.9) | |
|---|---|---|---|---|
| 08-4-0-0 | [μm] | [μm] | [μm] | Residual |
| MW | 1.35 | 3.09 | 5.85 | 0.98 |
| s | 0.01 | 0.01 | 0.01 | 0.05 |
| RSD | 0.54 | 0.25 | 0.14 | 5.04 |

### “Coarse Zeolite Powder”

| | d (0.1) | d (0.5) | d (0.9) | |
|---|---|---|---|---|
| | [μm] | [μm] | [μm] | Residual |
| MW | 1.63 | 9.51 | 35.66 | 0.56 |
| s | 0.00 | 0.05 | 0.24 | 0.02 |
| RSD | 0.15 | 0.56 | 0.66 | 2.93 |

The “ultrafine zeolite powder” therefore corresponds to the lower range of the invention, the “standard zeolite powder” to the upper range of the invention, and the “coarse zeolite powder” has about 50% of its mass situated outside (above) the range of the invention.

The experimental procedure was as follows:

Candida albicans was cultured, in accordance with methods frequently used in routine practice, in liquid culture (Sabouraud broth). Following determination of the cell count using a Thoma counting chamber, 12 experimental cultures were produced each with 1×10⁵ cells/ml, with no zeolite powder being added to 3 cultures (control), zeolite powder with the standard size distribution in an amount of 10 mg/ml being added to 3 cultures, ultrafine zeolite powder in an amount of 10 mg/ml being added to a further 3 cultures, and coarse zeolite powder in an amount of 10 mg/ml to 3 cultures more. The experimental cultures were incubated at 37° C. under aerobic conditions with shaking.

For the determination of the blank values, the experiment was also run with all of the media without microbes as well.

Over the period of 7 hours, aliquots of the cultures were taken at hourly intervals for determining the metabolic activity by a viability assay (resazurin assay). The resazurin assay is an assay frequently employed in biomedical research for measuring the cytotoxicity of substances. The redox indicator resazurin is added to the culture samples, and is reduced to form fluorescent resorufin by the NADH generated in the metabolism of the cells, the concentration of this resorufin being determined by fluorimetry [7].

FIG. 24 illustrates the results in graph form. It is clearly apparent that the effect of zeolite powder on the growth of the experimental microbe is heavily dependent on the particle size. Thus the experimental cultures with ultrafine zeolite powder (dotted curve, the lowermost curve in the right-hand region of the diagram) and standard zeolite powder (short-dashed curve, the next one above the ultrafine zeolite curve) exhibit significantly greater inhibition of metabolic performance than the coarse particle size fraction (long-dashed curve, the next in turn above). The uppermost curve, corresponding to the control group, reaches the exponential growth phase (which is also more strongly pronounced) at a much earlier stage.

FIG. 25 shows the measurements after 5, 6 and 7 h of incubation in the form of bar diagrams. Arranged in this case, in each case from left to right, are the coarse zeolite powder, the standard zeolite powder, the ultrafine zeolite powder, and the control group. As is clearly evident, the values for the coarse particle size fraction (far left) are closest to those of the controls without zeolite (far right).

In order to examine whether the different effect of the zeolite particle sizes used can also be demonstrated for other vaginal pathogens from the genus Candida, the following experiments were conducted on Candida glabrata [ATCC 2001], another microbe of vulvovaginal candidiasis:

The experimental procedure was as follows:

Candida glabrata was cultured, in accordance with methods frequently used in routine practice, in liquid culture (Sabouraud broth). Following determination of the cell count using a Thoma counting chamber, 12 experimental cultures were produced each with 6×10⁵ cells/ml, with no zeolite powder being added to 3 cultures (control), zeolite powder with the standard size distribution in an amount of 10 mg/ml being added to 3 cultures, ultrafine zeolite powder in an amount of 10 mg/ml being added to a further 3 cultures, and coarse zeolite powder in an amount of 10 mg/ml to 3 cultures more. The experimental cultures were incubated at 37° C. under aerobic conditions with shaking (110 rpm).

For the determination of the blank values, the experiment was also run with all of the media without microbes as well.

Over the period of 7 hours, aliquots of the cultures were taken at hourly intervals for determining the metabolic activity by a viability assay (resazurin assay) as described above.

The results are illustrated in graph form in FIG. 26. For C. glabrata as well it is clearly apparent that the growth-inhibiting effect of zeolite powder is strongly influenced by the particle size. Again, the experimental cultures with ultrafine zeolite powder (dotted curve, the second from bottom of the right-hand edge) and standard zeolite powder (short-dashed curve, at the very bottom on the right-hand edge) show significantly greater inhibition of metabolic performance than does the coarse particle size fraction (long-dashed curve, second curve from the top).

For better illustration of the results, FIG. 27 shows the measurement values after 5 and 6 h of incubation in the form of bar diagrams. The values of the coarse particle size fraction (bar on the far right in the diagram) are closest to those of the controls without zeolite (bar at the far left in the diagram). The standard zeolite growth groups are depicted on the inside left, the ultrafine zeolite groups on the inside right.

In order to underpin the results of investigation as set out above, a growth experiment as described before was carried out, but this time the number of seeded cells was reduced to about 1×10⁴ cells/ml, but with the incubation time extended to 16 h. As is apparent from FIG. 28, results were achieved which match very well the preceding results.

For further underpinning of the data determined experimentally, a growth experiment as described above was carried out, but this time with the active concentration of the zeolite powders used raised to 50 mg/ml and, in the form of the Vialight assay, with a second method employed for determining the metabolic activity. This assay is based on the quantification of the Adenosin triphosphate (ATP), generated by cells in their metabolism, through measurement of the bioluminescence which is emitted by the enzyme luciferase when the ATP is cleaved. [8]

As can be seen from the growth curves in FIG. 29, under these test conditions as well the standard product (short-dashed, lowermost curve) achieves a significantly greater growth-inhibiting effect than the coarse zeolite powder (long-dashed curve second from top).

If it is considered, in summary, that the “coarse zeolite powder” used also had notable fractions from the size range according to the invention, then there is a very good limit of the activity at the upper margin of the invention. The activity of the “ultrafine zeolite powder” reveals the limit of the lower margin, where in view of the smallness of the particles, the structure of the zeolite is presumably damaged or partly damaged.

The determination of the particle size is not critical, and is preferably accomplished using a Mastersizer 2000 from Malvern Instruments GmbH. This instrument operates on an optical basis according to the principle of diffraction of a laser beam. The intensity of the scattered light from a laser beam is measured, the beam penetrating a dispersed particle sample in continuous motion. Comparisons have shown that particle sizes measured accordingly correspond to those present in reality.

Particles which are present in a composition for assessment but are not within the stated and claimed particle size range are considered not to be present and are disregarded.

The substance according to the invention may be applied in any pharmaceutically acceptable composition; for instance, in particular, one or more of the following adjuvants may be used: pharmaceutically acceptable carrier materials; viable microorganisms and/or extracts thereof; nutrients for the healthy vaginal microbiota (e.g. lactose, etc.); substances which favorably influence the vaginal environment for the healthy vaginal microbiota, e.g. estradiol, organic acids, etc.

Administration may take place in particular in the form of creams, ointments, gels, suppositories, vaginal tablets, ovules, pessaries, foams, aerosols, powders, rinses, douches, suspensions, etc.

In both embodiments, the materials and methods which can be employed are known from the prior art and may be selected and implemented without problems by a pharmacist in knowledge of the disorder to be treated (application) and of the invention.

### REFERENCES

[1] Hainer, B., L. (2011) Vaginitis: Diagnosis and Treatment; American Academy of Family Physicians; http://www.sonoma.edu/users/w/wilkosz/n540a-07/VaginitisDX and TX.pdf
[2] AWMF online: S1 guidance: Bacterial vaginosis (BV) in gynecology and midwifery; AWMF guidance register No. 015/28; current version: July 2013.
[3] AWMF online: S2k guidance: Vulvovaginal candidiasis; AWMF register No. 015/072; version December 2013.
[4] Candida albicans DSM No. 1386 https://www.dsmz.de/catalogues/details/culture/DSM-1386.html?tx_dsmzresources_pi5%5BreturnPid %5D=304
[5] Gardnerella vaginalis DSM No. 4944 https://www.dsmz.de/catalogues/details/culture/DSM-4944.html?tx_dsmzresources_pi5%5BreturnPid %5D=304
[6] Lactobacillus crispatus DSM No. 20584 https://www.dsmz.de/catalogues/details/culture/DSM-20584.html?tx_dsmzresources_pi5%5BreturnPid %5D=304
[7] Rampersad, S., N. (2012) Multiple Applications of Alarm Blue as an Indicator of Metabolic Function and Cellular Health in Cell Viability Bioassays; Review, Sensors 2012, 12, 12347-12360.
[8] Vialight™ MDA plus—High sensitivity microbial detection kit with extended signal stability; Lonza LT07-122.
[9] Protocol for microbiological investigation S0811-01
[10] Protocol for microbiological investigation S0903-19
[11] Lactobacillus casei DSM No. 20011 https://www.dsmz.de/catalogues/details/culture/DSM-20011.html?tx_dsmzresources_pi5%5BreturnPid %5D=304
[12] Lactobacillus jensenii DSM No. 20557 https://www.dsmz.de/catalogues/details/culture/DSM-20557.html?tx_dsmzresources_pi5%5BreturnPid %5D=304
[13] Enzytec™ D/L Lactic acid Art. No. E1255—Usage instructions; r-Biopharm.
[14] Candida glabrata ATCC No. 2001 https://www.atcc.org/˜/ps/2001.ashx

1-4. (canceled)

5. A method of treating vaginitis in a mammal, comprising applying a therapeutically effective amount of a pharmaceutically acceptable composition of clinoptilolite having a particle size of between 0.2 and 10 μm.

6. The method of claim 5, wherein applying the composition of clinoptilolite includes externally applying the composition of clinoptilolite.

7. The method of claim 5, wherein the composition of clinoptilolite is substantially free of heavy metals.

8. The method of claim 5, wherein the method is used to treat one or more of bacterial vaginosis, vulvovaginal candidiasis, and trichomoniasis.

9. The method of claim 5, wherein the method includes treating vaginitis in a human.

10. The method of claim 5, wherein the composition of clinoptilolite applied to the mammal includes one or more pharmaceutically acceptable adjuvants.

11. The method of claim 10, wherein the composition of clinoptilolite applied to the mammal includes one or more of a pharmaceutically acceptable carrier material, a viable microorganisms, a microorganism extract, a nutrient for healthy vaginal microbiota, and a substances which favorably influences the vaginal environment for healthy vaginal microbiota.

12. The method of claim 11, wherein the composition of clinoptilolite applied to the mammal includes one or more of lactose, estradiol, and an organic acid.

13. The method of claim 5, wherein applying the composition of clinoptilolite includes applying a foam, a suppository, a vaginal tablet, an ovule, a gel, an aerosol, a powder, a rinse, a douche, a cream, an ointment, or a suspension.

14. A method of restoring a healthy vaginal microbiota in a mammal, comprising applying a therapeutically effective amount of a pharmaceutically acceptable composition of clinoptilolite having a particle size of between 0.2 and 10 μm.

15. The method of claim 14, wherein applying the composition of clinoptilolite includes externally applying the composition of clinoptilolite.

16. The method of claim 14, wherein the composition of clinoptilolite is substantially free of heavy metals.

17. The method of claim 14, wherein the method includes applying the composition of clinoptilolite to a human.

18. The method of claim 14, wherein the composition of clinoptilolite applied to the mammal includes one or more adjuvants.

19. The method of claim 14, wherein the composition of clinoptilolite applied to the mammal includes one or more of a pharmaceutically acceptable carrier material, a viable microorganisms, a microorganism extract, a nutrient for healthy vaginal microbiota, and a substances which favorably influences the vaginal environment for healthy vaginal microbiota.

20. The method of claim 14, wherein applying the composition of clinoptilolite includes applying a foam, a suppository, a vaginal tablet, an ovule, a gel, an aerosol, a powder, a rinse, a douche, a cream, an ointment, or a suspension.

21. A pharmaceutically acceptable composition of clinoptilolite comprising particles sized between 0.2 and 10 μm for use in the treatment of vaginitis in mammals, and for restoring a healthy vaginal microbiota.

22. The pharmaceutically acceptable composition of claim 21, wherein the composition is substantially free of heavy metals.

23. The pharmaceutically acceptable composition of claim 21, further comprising one or more pharmaceutically acceptable carriers or pharmaceutically acceptable adjuvants.

24. The pharmaceutically acceptable composition of claim 21, consisting essentially of clinoptilolite particles between 0.2 and 10 μm in size.
